(19) United States
(12) Patent Application Publication
(10) Pub. No.: US 2024/0315823 A1
(43) Pub. Date: Sep. 26, 2024

(54) SURGICAL MATERIAL

(71) Applicant: TISSIUM SA, Paris (FR)

(72) Inventors: Miguel LOPES, Issy les Moulineaux (FR); Maria PEREIRA, Lisbon (PT); David MOREAU, Paris (FR); Elisa BITTON, Paris (FR)

(21) Appl. No.: 18/261,534

(22) PCT Filed: Jan. 17, 2022
(86) PCT No.: PCT/EP2022/050924

§ 371 Date: Jul. 14, 2023

Related U.S. Application Data
(60) Provisional application No. 63/138,743, filed on Jan. 18, 2021.

(51) Int. Cl. A61F 2/00; A61L 24/00; A61L 24/04; A61L 31/06; A61L 31/14

(52) U.S. Cl. CPC A61F 2/0063; A61L 24/001; A61L 24/046; A61L 31/06; A61L 31/14; A61F 2230/0006

(57) ABSTRACT

The present disclosure relates to a novel surgical material comprising mesh and a polymer composition, and its use in treating a hernia and related diseases and conditions. The present disclosure also relates to a method of manufacturing the surgical material.

### FIELD OF THE DISCLOSURE

The present invention relates to a surgical material comprising a polymer composition, and its use in treating a hernia and any diseases and conditions requiring to provide support to weakened, abnormal or damaged tissue. The present disclosure also relates to a method of manufacturing the surgical material.

### BACKGROUND OF THE DISCLOSURE

A hernia is a protrusion of an organ through the cavity which normally contains it. For treating a hernia, a mesh can be placed over the weakened region of tissue. The mesh may be fixed in the region using tacks, sutures, self-fixating meshes or adhesives. The currently available fixation techniques have drawbacks, such as poor fixation strength, pain, poor usability and/or tissue damages. Moreover, sutures are difficult and time consuming to place in minimally invasive procedures. The mesh can be placed at different location in between the layers of the abdominal wall, and the mesh location may depend on the surgeon's habit and patient history.

Frequently the mesh is placed between two layers of tissue suggesting lower fixation requirements. However, in some specific procedures such as intraperitoneal mesh placement (IPOM), the mesh is placed at the abdominal cavity in contact with only one layer of tissue, requiring higher fixation strength. Tackers can be resorbable (e.g., PGLA) or not (e.g., titanium). Despite being quite easy to perform, IPOM is associated with a higher risk of undesired adhesions creation to the viscera (Gungor et al., 2010) and higher pain due to the fixation method (usually 1 to 2 rows of tacks, sometimes complemented with transfascial sutures).

The choice of the fixation methods depends on the surgeon's habits, the hernia location, the chosen approach, and the patient. In the inguinal scenario, most of the time minimal fixation is required resulting in high adoption of self-fixating meshes (ProGrip™-Medtronic), a few tacks or even fibrin glue. When approaching the hernia through an open procedure, sutures are used most of the time.

Several fixation methods currently exist to fix the mesh on to the tissue: tacks, sutures and some surgical glues made of fibrin or cyanoacrylate. However, they all suffer from at least one of the following drawbacks: pain¹, adhesion^{2, 3} or poor performance (i.e. fixation strength) and/or usability.

Several limitations to tacks including but not limited to: (1) this fixation is penetrating causing both acute and/or chronic post-operative pain (2) tacks can be responsible for visceral attachment (3) depending on the hernia location, there is a risk of misfiring the tissue because of inadequate deployment angle (some require a 90° angle and counterpressure application to maximize tacks penetration) (4) they do not allow repositioning of the mesh once deployed (5) while applying tacks, the mesh can drift off center and it is difficult to obtain flat mesh positioning. Despite all those limitations, tacks remain the standard of care of the IPOM fixation in the absence of more suitable alternatives.

Different adhesives have been/are being tested in the context of intraperitoneal placement but are not yet optimal and widely adopted: (1) fibrin—despite its use in inguinal and some ventral hernia procedure, it failed to show convincing clinical performance in IPOM repair likely because of too low fixation strength. (2) cyanoacrylate-based glues are sometimes used for inguinal repair, but adoption is for now limited likely due to the poor usability (i.e. reactive with body fluids) and known tissue toxicity and impairment of cell ingrowth.^{4,5} For both compounds, an in situ application to the mesh is required—no mesh precoating possible due to self-polymerization—, which does not allow standardization. Furthermore, both are low viscosity and can drip to the surrounding tissue. The uncontrolled self-polymerization and dripping to surrounding tissue does not allow to reposition of the mesh if needed.

Therefore, there remains an unmet medical need for new surgical mesh and fixation methods to fix the mesh on to the tissue for patients with hernia, especially those who undergo an IPOM procedure.

### SUMMARY OF THE DISCLOSURE

In some embodiments, a surgical material comprises a polymer composition applied to any substrate having a surface, more particularly a tissue repair support, such as surgical patch, for instance a mesh substrate, wherein the polymer composition has a post-it effect allowing the surgical material to be positioned and repositioned on body tissue during surgery and wherein the polymer composition can be activated after positioning on the body tissue to attach the surgical material to the tissue.

In some embodiments, the polymer composition is a composition described in PCT/EP2020/079941. In some embodiments, the polymer composition is activated by light. In some embodiments, the mesh is a circular shape, and wherein a ratio of a weight of the polymer composition to the total polymer pattern length (e.g. perimeter of the inner crown plus perimeter of the outer crown) is from about 0.03 g/cm to about 0.08 g/cm, more particularly from about 0.04 g/cm to about 0.06 g/cm.

In some embodiments, a method of treating a hernia comprises, i) positioning a surgical material over a herniated defect, and wherein the surgical material preferably comprises a polymer composition such as, for example, described in PCT/EP2020/079941 (incorporated herein by reference), and ii) activating the polymer composition to attach the surgical material to tissue within a body proximate the herniated defect. In some embodiments, the method may further comprise repositioning the surgical material as necessary, after step i) and before step ii). In some embodiments, the polymer composition may be activated by light during step ii).

In some embodiments, a method of manufacturing a surgical material may comprise applying a polymer composition such as, for example, described in PCT/EP2020/079941 on a mesh substrate, wherein the polymer composition can be activated during surgery to attach the surgical material to tissue within a body.

### BRIEF DESCRIPTION OF THE FIGURES

The accompanying drawings, which are incorporated in and constitute a part of this specification, illustrate the disclosed embodiments and, together with the description, explain the principles of the disclosed embodiments. In the drawings:

FIG. 1 illustrates the different layers of the abdominal wall where a mesh can be placed.⁶

FIG. 2 illustrates knitting tightness of two commercially available composite meshes, Ventralight™ ST and Symbotex™.

FIG. 3 [reserved].

FIG. 4 illustrates implantations of a surgical material of the present disclosure compared to Absorbatacks™ in a pig model.

FIG. 5 illustrates an exemplary surgical material of the present disclosure.

FIG. 6 illustrates a comparison of acute burst results of a surgical material having double crown pattern to a surgical material having a full coating pattern.

FIG. 7 illustrates burst acute performance of surgical materials of the present disclosure in two polymer quantity ratios.

FIG. 8 illustrates a set-up for a lap shear test.

FIG. 9 illustrates a set up for a burst ball test.

FIG. 10 illustrates acute lap shear performance of a surgical material of the present disclosure in comparison to fibrin.

FIG. 11 illustrates overview of burst ball acute performance using different meshes.

FIG. 12 illustrates a study design of a chronic animal test.

FIG. 13 illustrates appearance of meshes within 90 days of the implantation. The first row shows a surgical material of the present disclosure, and the second row shows Absorbatacks™.

FIGS. 14-15 [reserved].

FIG. 16 illustrates tissue tolerance and cell ingrowth of the mesh coated with the polymer composition (surgical material) or fixed with Absorbatacks™.

FIG. 17(a) illustrates a burst ball set up. FIG. 17(b) illustrates burst ball test results at 3 months of a surgical disclosure of the present disclosure compared to Absorbatacks™.

FIG. 18(a) illustrates a T-peel test set up. FIG. 18(b) illustrates ingrowth strength of a surgical material of the present invention compared to Absorbatacks™.

FIG. 19 shows chronic study overview, demonstrating equivalent performance between a polymer composition, POL004, and Absorbactacks™.

FIG. 20 shows acrylate conversion rates for the polymer composition when using different meshes.

FIG. 21(a)-(c) illustrates exemplary ratios and/or weight calculations based on specific mesh and/or polymer pattern.

FIG. 22 illustrates burst ball test results at 3 months of POL004 compared to SorbaFix™ fixed to Ventralight™.

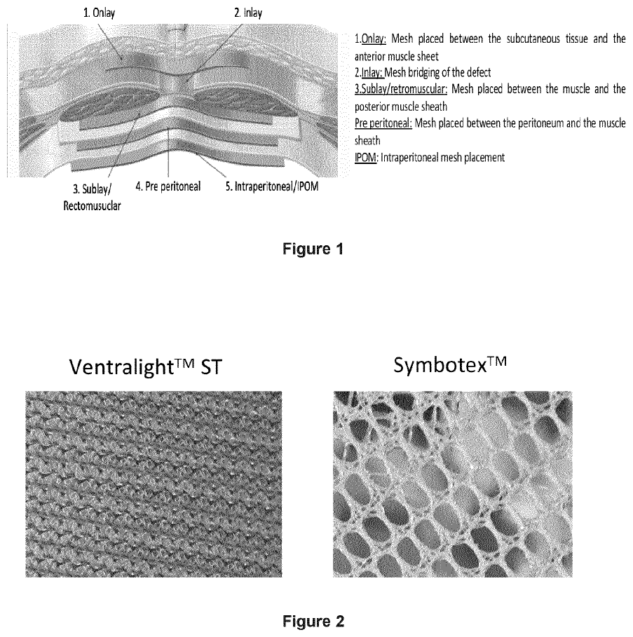

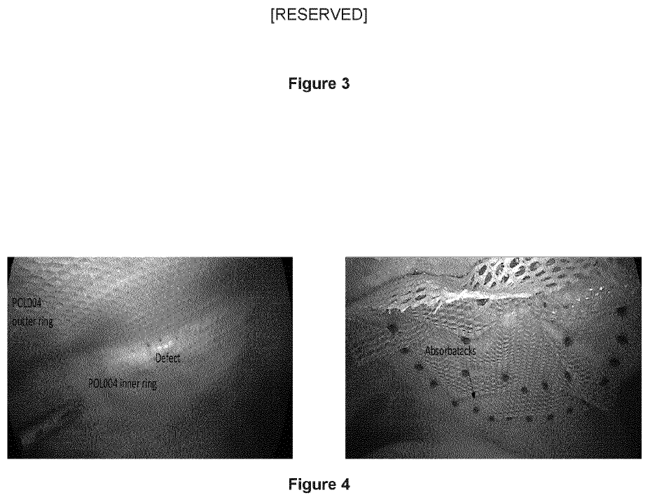

### DETAILED DESCRIPTION OF THE DISCLOSURE

### A Polymer Composition

In some embodiments, “a polymer composition” refers to any polymer compositions such as, for example, described in PCT/EP2020/079941, the contents of which are herein incorporated by reference. In some embodiments, “a polymer composition” refers to any polymer compositions described in U.S. application Ser. No. 15/737,103 based on PCT/EP2016/064015, U.S. application Ser. No. 15/737,143 based on PCT/EP2016/064016, or WO2019/180208, the contents of which are herein incorporated by reference. In some other embodiments, “a polymer composition” refers to any polymer compositions described in U.S. Pat. Nos. 7,722,894 and 8,143,042, 10,179,195, 9,724,447, U.S. application Ser. No. 16/206,937, or EP3005221, the contents of which are herein incorporated by reference. According to some preferred embodiments, suitable polymer is selected from the group consisting of poly(glycerol sebacate acrylate) or derivative thereof such as for example aminated PGSA (WO2021/078962). According to a preferred embodiment, said polymer is a poly(glycerol sebacate acrylate) derivative having following structure:

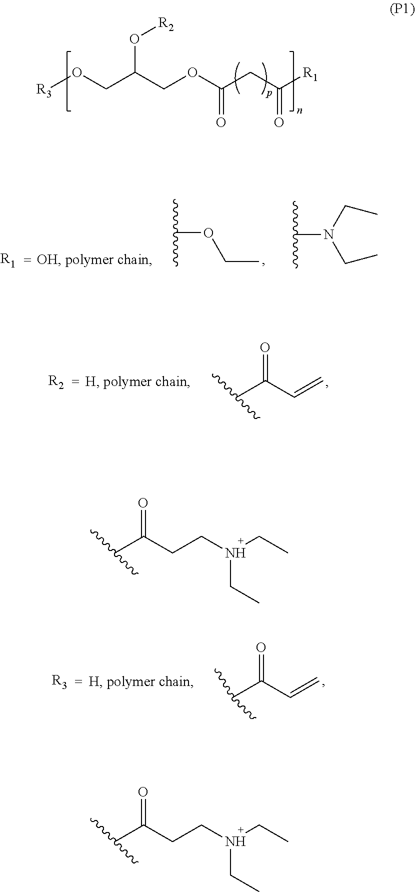

In the above structure, n and p, independently from each other, can be an integer equal to or greater than 1.

In some embodiments, “a polymer composition” refers to an adhesive composition that is a light-curable compound. “Light curable compound” refers to compounds that are configured to polymerize or otherwise cure upon receiving appropriate radiant energy, more particularly in the form of light from a light source. According to preferred embodiment, said light is visible light, more preferably visible blue light.

The light-curable compound may comprise a pre-polymer and a photoinitiator, said photoinitiator being able to induce polymerization of the said pre-polymer when exposed to light of a specific wavelength.

According to at least one embodiment, said photoinitiator is sensitive to ultraviolet (UV) radiations.

According to at least one embodiment, said photoinitiator is sensitive to radiations with a 405 nm wavelength.

In some embodiments, the polymeric backbone of the pre-polymer comprises a polymeric unit of the general formula (-A-B-)n, wherein A is derived from a substituted or unsubstituted polyol or mixture thereof and B is derived from a substituted or unsubstituted polyacid or mixture thereof; and n represents an integer greater than 1. The polymeric backbone is made up of repeating monomer units of general formula-A-B-. The term “substituted” has its usual meaning in chemical nomenclature and is used to describe a chemical compound in which a hydrogen on the primary carbon chain has been replaced with a substituent such as alkyl, aryl, carboxylic acid, ester, amide, amine, urethane, ether, or carbonyl. Component A of the pre-polymer may be derived from a polyol or mixture thereof, such as a diol, triol, tetraol or greater. Suitable polyols include diols, such as alkane diols, preferably octanediol; triols, such as glycerol, trimethylolpropane, trimethylolpropane ethoxylate, triethanolamine; tetraols, such as erythritol, pentaerythritol; and higher polyols, such as sorbitol. Component A May 5 also be derived from unsaturated polyols, such as tetradeca-2,12-diene-1,14-diol, polybutadienediol or other polyols including macromonomer polyols such as, for example polyethylene oxide, polycaprolactone triol and N-methyldiethanoamine (MDEA) can also be used. Preferably, the polyol is substituted or unsubstituted glycerol. Component B of the pre-polymer is derived from a polyacid or mixture thereof, preferably diacid or triacid. Exemplary acids include, but are not limited to, glutaric acid (5 carbons), adipic acid (6 carbons), pimelic acid (7 carbons), sebacic acid (8 carbons), azelaic acid (nine carbons) and citric acid. Exemplary long chain diacids include diacids having more than 10, more than 15, more than 20, and more than 25 carbon atoms. Non-aliphatic diacids can also be used. For example, versions of the above diacids having one or more double bonds can be used to produce polyol-diacid copolymers. Preferably the polyacid is substituted or unsubstituted sebacic acid.

Examples of suitable photoinitiators sensitive to UV radiations include, but are not limited to: 2-dimethoxy-2-phenyl-acetophenone, 2-hydroxy-1-[4-(hydroxyethoxy)phenyl]-2-methyl-1-propanone (Irgacure 2959), 1-hydroxycyclohexyl-1-phenyl ketone (Irgacure 184), 2-hydroxy-2-methyl-1-phenyl-1-propanone (Darocur 1173), 2-benzyl-2-(dimehylamino)-1-[4-morpholinyl) phenyl]-1-butanone (Irgacure 369), methylbenzoylformate (Darocur MBF), oxy-phenyl-acetic acid-2-[2-oxo-2-phenyl-acetoxy-ethoxy]-ethyl ester (Irgacure 754), 2-methyl-1-[4-(methylthio)phenyl]-2-(4-morpholinyl)-1-propanone (Irgacure 907), diphenyl(2,4,6-trimethylbenzoyl)-phosphine oxide (Darocur TPO), phosphine oxide, phenyl bis(2,4,6-trimethyl benzoyl) (Irgacure 819), and combinations thereof.

According to another embodiment, said photoinitiator is sensitive to visible light (typically blue light or green light).

Examples of photoinitiators sensitive to visible light include, but are not limited to: diphenyl(2,4,6-trimethylbenzoyl)-phosphine oxide, eosin Y disodium salt, N-Vinyl-2-Pyrrolidone (NVP) and triethanolamine, and camphorquinone.

According to some embodiments, the polymer composition further comprises bioactive agent (e.g., antibiotic, tissue growth factor, . . . ).

According to some embodiments, the viscosity of the polymer composition is 500 to 100,000 cP, more preferably 1,000 to 50,000 cP, even more preferably 2,000 to 40,000 cP and most preferably 2,500 to 25,000 cP. Viscosity analysis is performed using a Brookfield DV-II+Pro viscosimeter with a 2.2 mL chamber and SC4-14 spindle, the speed during the analysis is varied from 5 to 80 rpm. The above-mentioned viscosity is present in the relevant temperature range for medical application i.e. room temperature up to 40° C., preferably 37° C.

### A Surgical Material

In some embodiments of the present disclosure, a surgical material comprises a mesh coated with a polymer composition, such as a polymer described, for example, in PCT/EP2020/079941. In some other embodiments, a polymer composition may be ones described in other patent applications that are herein incorporated by reference. In some other embodiments, a polymer composition may be any polymer compositions having adhesive and/or sealant properties.

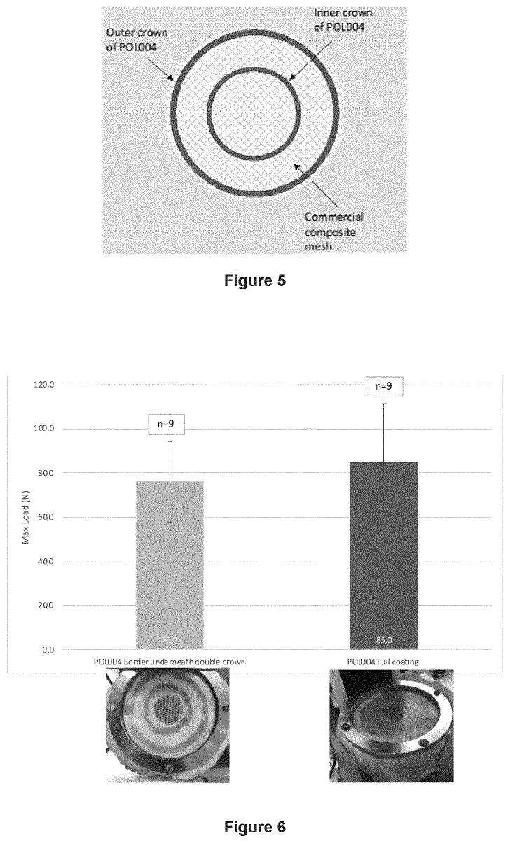

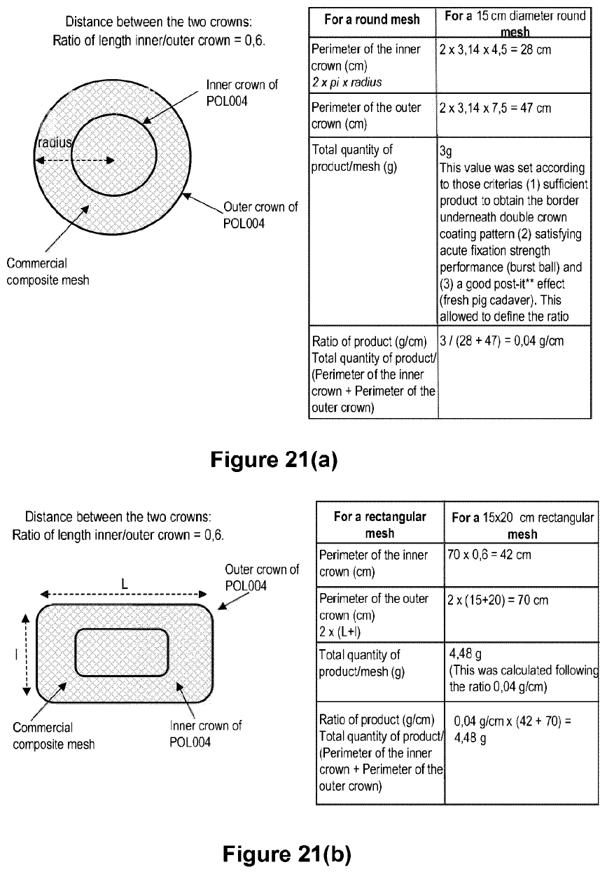

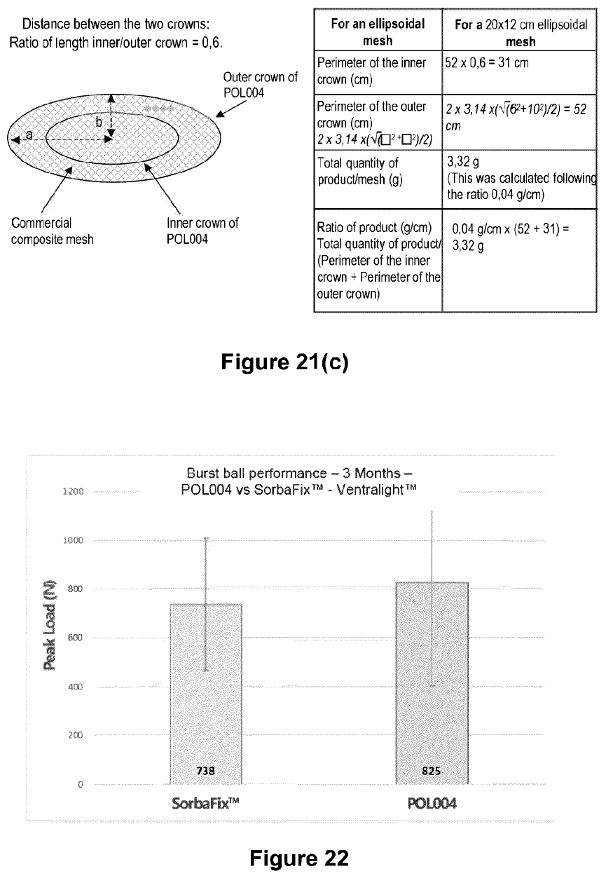

In some embodiments, depending on the mesh reference, a ratio of the weight of applied polymer composition to the total polymer pattern length (e.g. perimeter of the inner crown plus perimeter of the outer crown) can be between about 0.03 g/cm and about 0.08 g/cm, more particularly between about 0.04 g/cm and about 0.06 g/cm. In other embodiments, the ratio can be 0.03 g/cm, 0.035 g/cm, 0.04 g/cm, 0.045 g/cm, 0.05 g/cm, 0.055 g/cm, 0.06 g/cm, 0.065 g/cm, 0.07 g/cm, or above. FIG. 21 illustrates particular ratio and/or polymer composition weight determination based on specific mesh and/or polymer pattern. The quantity of polymer composition may depend on the mesh composition. For example, when a mesh's knitting is tight, more polymer composition may be applied on a mesh compared to when a mesh's knitting is loose. As illustrated in FIG. 5, the coating may be applied following a certain pattern. Alternatively, the coating may be applied on the entire surface of the mesh. In at least one embodiment, the coating may be applied in any various patterns. For example, a polymer composition may be coated in dots rather than lines. According to some embodiments, the coating may be applied on circular, oval, or rectangular mesh and pattern may be coated according to circular, oval, or rectangular patterns.

In some embodiments, a polymer composition is applied following a double crown pattern. The outer crown of the coating pattern may allow a smooth continuity between the mesh and the tissue, minimizing the risk of mesh detachment and viscera attachment. In some embodiments, the inner ring may be used because, for example, (1) it provides a homogeneous fixation over the mesh and maximizes the contact between the mesh and the targeted tissue (2) it reinforces theoretically the area that is closer to the defect.

In some embodiments, when a polymer composition is applied following a double crown pattern, the distance between the two crowns can be randomly chosen. In some embodiments, a ratio of the inner crown length to the outer crown length can be about 0.45, 0.5, 0.55, 0.6, 0.65, 0.7, 0.75 or 0.8. In some embodiment, the length ratio between the outer crown and the inner crown may be from about 0.55 to about 0.65. In some embodiment, the length ratio between the outer crown and the inner crown may be from about 0.6.

In some embodiments, the coating pattern includes only the outer ring. In some other embodiments, the coating pattern may include the outer ring and any additional patterns within the outer ring.

A surgical material may be used to treat hernia and any diseases and conditions requiring to provide support to weakened, abnormal or damaged tissue (for example tissues with a hole, such as fistulas). In some embodiments, a surgical material is a mesh pre-coated with a polymer composition. After the surgical material is positioned over a defect by a doctor, the polymer composition may be activated, for example, using light. According to some embodiments, this light is visible light, more preferably visible blue light. In some embodiments, light may be provided via an endoscope and a LED module. In some embodiments, the diameter of the endoscope can be about 3 mm, 4 mm, 5 mm, 6 mm, 7 mm, 8 mm, 9 mm, 10 mm, 11 mm, 12 mm, or 13 mm. In some embodiments, the endoscope can have about 10 mm diameter. In some embodiments, the LED module can have an optical power of about 5 W, 6 W, 7 W, 8 W, 9 W, 10 W, 11 W, 12 W, 13 W, 14 W, or 15 W. For example, the LED module can have an optical power of about 10 W.

### Advantages of a Surgical Material

In some embodiments of the present disclosure, a surgical material comprises a mesh coated with a polymer composition. The surgical material has several advantages.

### Non Penetrating Fixation:

Unlike tacks, a polymer composition may be non penetrating suggesting that it would result in lower post-operative pain due to mesh fixation, which should be accepted by surgeons and experts and should benefit patients. Furthermore, non-penetrating fixation does not damage the mesh or surgical substrate, potentially decreasing the prevalence of adhesions in the case of intra-abdominal use.

### Easy to Use and Easy to Deploy—Post-it Effect:

The polymer composition's viscosity allows it to be applied on the mesh before rolling and insertion in the abdominal cavity. This is considerably simpler, more standardized (similar pattern and polymer quantity) and faster than application in situ as proposed in prior methods (inside the abdominal wall) such as the use of human fibrin adhesive (for example, Tissucol/Tisseel), fibrin glue (Baxter Healthcare, Deerfield, IL, USA) or cyanoacrylaye adhesives. These tissue adhesives are generally applied by the surgeon on throughout the mesh as dots using a laparoscopic applicator.

Once the mesh and a polymer composition are in the intraperitoneal space, the post-it effect allows an easy positioning and repositioning of the mesh over the defect. The post-it effect is the ability of the polymer composition to provide stickiness of the mesh to the tissue wall before it is firmly fixed to the tissue via activation, providing the surgeon with, for example, the ability to reposition the mesh if so desired during surgery. This post-it effect is due to the polymer composition's degree of stickiness before light activation. The post-it effect allows for proper centering/positioning of the mesh over the defect to avoid or reduce hernia recurrence, which is one of the risks for the surgeon. The term “repositioning” or “reposition” also includes meanings of “adjust mesh positioning” and/or “readjustment”.

### On Demand Activation:

A surgical material may be activated once it is positioned over a defect. Because a doctor may decide when to activate the polymer composition, the on-demand activation provides more control over the mesh positioning. In some embodiments, the polymer composition may be activated by light, for example, a 405 nm LED light. In some other embodiments, the polymer composition may be activated by a laparoscopic solution.

### Fully Sealing Solution:

FIG. 4 illustrates implantations of a surgical material of the present disclosure compared to a mesh fixed with Absorbatacks™ in a pig model. As illustrated in FIG. 4, having a polymer composition on the mesh borders allow to obtain a very smooth transition between the mesh and the tissue, which is not the case for tacks, even when placed using the double crown pattern as it is displayed in FIG. 4. The surgical material composition is also better affixed to the targeted tissue, showing less wrinkles than than the mesh fixed with Absorbatacks™

### A Method of Treating a Hernia or Similar Conditions

In some embodiments, a surgical material of the present disclosure may be used to treat a hernia or similar conditions such as any diseases and conditions requiring to provide support to weakened, abnormal or damaged tissue (for example tissues with a hole, such as fistulas). The surgical material may serve as scaffold for cell to grow aiming to reinforce the damaged region. A surgical material of the present disclosure comprises a mesh and a polymer composition. In some embodiments, a polymer composition may be pre-coated on a composite mesh before it is shipped to a medical facility, such as a hospital. But in at least one embodiment, a doctor may apply a polymer composition on to a mesh on-site before its implantation.

In some embodiments, a surgical material of the present disclosure may be used to treat a hernia or similar conditions. In some embodiments, a surgical material of the present disclosure may be used for the IPOM. In some other embodiments, the surgical material may be used to treat other similar indications and placed in various locations, for example, the ventral retromuscular or inguinal procedures. In some embodiments, the surgical material may be used to treat a human or an animal.

In some embodiments, a surgical material may be positioned over a defect by a doctor. The surgical material has a post-it effect, due to its degree of stickiness before activation. Thus, in some embodiments, doctors may reposition the surgical material to a proper location as necessary. Once the surgical material is properly positioned over a defect, a polymer composition may be activated, for example, by light or a laparoscopic solution. In at least one embodiment, light may be provided via an endoscope and a LED module. In some embodiments, the diameter of the endoscope can be about 3 mm, 4 mm, 5 mm, 6 mm, 7 mm, 8 mm, 9 mm, 10 mm, 11 mm, 12 mm, or 13 mm. In some embodiments, the endoscope can have about 10 mm diameter. In some embodiments, the LED module can have an optical power of about 5 W, 6 W, 7 W, 8 W, 9 W, 10 W, 11 W, 12 W, 13 W, 14 W, 15 W, or above. For example, the LED module can have an optical power of about 10 W.

### A Method of Manufacturing a Surgical Material

In some embodiments, a surgical material of the present disclosure comprises a polymer composition, such as a polymer described in PCT/EP2020/079941, coated on a composite mesh. In some other embodiments, a polymer composition may be ones described in other patent applications that are herein incorporated by reference. In some other embodiments, a polymer composition may be any polymer compositions having adhesive and/or sealant properties.

In some embodiments, the coating of a polymer composition can be applied using a coating device, which may allow a standardization of the surgical material product. In some embodiments, a polymer composition may be pre-coated on a mesh, such as a composite mesh, before it is shipped to a medical facility, such as a hospital. But in at least one embodiment, a doctor may apply a polymer composition on to a mesh on-site before its implantation.

In some embodiments, depending on the mesh reference, a ratio of the weight of applied polymer composition to the total polymer pattern length (e.g., perimeter of the inner crown plus perimeter of the outer crown) can be from about 0.03 g/cm to about 0.08 g/cm, more particularly from about 0.04 g/cm to about 0.06 g/cm. In other embodiments, the ratio can be 0.03 g/cm, 0.035 g/cm, 0.04 g/cm, 0.045 g/cm, 0.05 g/cm, 0.055 g/cm, 0.06 g/cm, 0.065 g/cm, 0.07 g/cm, or above. The quantity of polymer composition may depend on the mesh composition. When a mesh's knitting is tight, more polymer composition may be applied on a mesh compared to when a mesh's knitting is loose.

As illustrated in FIG. 5, the coating may be applied following a certain pattern. Alternatively, the coating may be applied on the entire surface of the mesh. In at least one embodiment, the coating may be applied in any various patterns. For example, a polymer composition may be coated in dots rather than lines.

In some embodiments, a polymer composition is applied following a double crown pattern. When a polymer composition is coated following a double crown pattern, the distance between the two crowns can be randomly chosen. In some embodiments, a ratio of an inner crown length to an outer crown length can be about 0.45, 0.5, 0.55, 0.6, 0.65, 0.7, 0.75 or 0.8. In some embodiment, the length ratio between the outer crown and the inner crown may be from about 0.55 to about 0.65. In some embodiment, the length ratio between the outer crown and the inner crown may be from about 0.6.

In some embodiments, the coating pattern includes only the outer ring. In some other embodiments, the coating pattern may include the outer ring and any additional patterns within the outer ring.

The surgical material may be used to treat a hernia or other similar conditions. In some embodiments, a surgical material is positioned over a defect by a doctor. The surgical material has a post-it effect, due to its stickiness before activation. Thus, in some embodiments, doctors may reposition the surgical material to a proper location, as necessary. Once the surgical material is properly positioned over a defect, a polymer composition may be activated, for example, by light or a laparoscopic solution.

### EXAMPLES

The examples are provided below further illustrate and exemplify various testings using a surgical material of the present disclosure. It is to be understood that the scope of the present disclosure is not limited in any way by the scope of the following examples.

The following abbreviations have the definitions set forth below:
1. IPOM: Intraperitoneal mesh onlay placement
2. ePTFE: Expended Polytetrafluoroethylene
3. IFU: Instruction For Use
4. HA: Sodium Hyaluronate
5. PGA: Polyglycolic Acid
6. PGLA: Poly Lactic-Co-Glycolic Acid
7. PEG: Polyethylene Glycol
8. CMC: Carboxymethyl Cellulose

### Example 1. Testing on Three Commercial Composite Meshes

A surgical material of the present invention comprises a polymer composition applied on to a mesh. A mesh is characterized by different aspects: material used, pore size, weight and elasticity among other. Meshes can be synthetic, biosynthetic or biologic. Synthetic ones are mainly made of polyester, polypropelene and ePTFE. Biologic ones are preferred in contaminated field. When the mesh is not put in contact with the viscera (inguinal and ventral procedure other than IPOM), the use of non-coated synthetic meshes is advised. In the context of IPOM, composite meshes are used as they are placed in contact with the viscera. They are made of two sides: a non-absorbable side that promotes tissue ingrowth and an absorbable (or non-absorbable side depending on the supplier) that prevents adhesion creation between the mesh and the viscera.

Preclinical tests were focused on 3 commercial composite meshes looking at the three main products: Symbotex™ from Medtronic, Ventralight™ ST from Bard, Proceed® from Ethicon. Details of the make up of resorbable fixations of these three products can be found in Annex 1.

### Medtronic—Symbotex™:

The Medtronic mesh is a polyester based mesh coated with collagen and glycerol. The mesh is highly translucent (allows light to go through for the activation step when used with a polymer composition, POL004, see below), easy to handle even after hydration (not too soft, not too rigid) and present a bioresorbable coating that remains in place even after manipulation.

### Ethicon—Proceed®:

Regarding Ethicon mesh, it is made of polypropylene and coated with oxidized cellulose. The mesh is less translucent than other composite meshes, requiring adapted light activation conditions when combined with the polymer composition (e.g., time, intensity). This mesh does not require hydration.

### Bard—Ventralight™ ST:

The Bard mesh is made of polypropylene and PGA coated with sodium hyaluronate (HA), carboxymethylcellulose (CMC) and polyethylene glycol (PEG) based hydrogel. After hydration the mesh is very soft, which makes it challenging to maintain the mesh in place while fixing with tacks.

A polymer composition (disclosed in PCT/EP2020/079941, and hereby referred to as “POL004”) is applied on the three commercial comspite meshes, and their performances were tested. POL004 is a poly(glycerol sebacate acrylate) derivative:

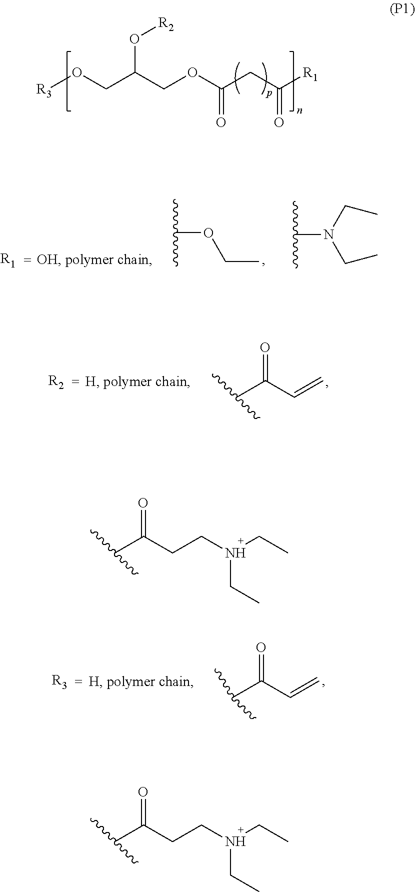

In the above structure, n and p, independently from each other, can be an integer equal to or greater than 1.

As illustrated in FIG. 2, mesh knitting's tightness varies, and this may affect the surgical material product's performance and may require adjustments to the polymer quantity required.

### Example 2. Testing on Coating Pattern

Different coating patterns were tested ex vivo (with the Symbotex™) comparing a full coating-product all over the mesh to a double crown coating, as illustrated in FIG. 5.

FIG. 6 illustrates an acute burst test results of a surgical material when a polymer composition, POL004, was applied in a double crown pattern compared to when it was applied on the entire mesh. As illustrated in FIG. 6, the test result performances of the two coating patterns were similar.

Therefore, the border underneath double crown coating pattern may be used to minimize product quantity and reduce the polymer quantity ratio.

### Example 3. Testing on the Quantity of a Polymer Composition

The following criteria were used to define the required product quantity: (1) sufficient product to obtain the border underneath double crown coating pattern, (2) satisfying acute fixation strength performance (quantified using the burst ball set up), and (3) a good post-it effect (quantified using a fresh pig cadaver model).

### Symbotex™

The first mesh that was tested in combination with the polymer composition, POL004, was the Symbotex™ (Medtronic).

The quantity of product that was used for this mesh was empirical following the criteria mentioned above. A ratio of 0.04 g/cm (corresponding to 3 g for a circular mesh of 15 cm diameter using the border underneath double crown coating pattern) was reached. As good performance was observed using that product quantity.

### Proceed®

For the Proceed® mesh, we used the same quantity of POL004 that was used for the Symbotex™ mesh.

### Ventralight™ ST

For the Ventralight™ ST mesh the polymer may interlock in the mesh knitting, as illustrated in FIG. 2. With this mesh, the POL004's quantity was increased to reach similar performance to the surgical material when using a Symbotex™ mesh. It was concluded that a ratio of 0.055 g/cm was sufficient for this mesh to obtain a satisfying post-it effect and sufficient fixation strength performance.

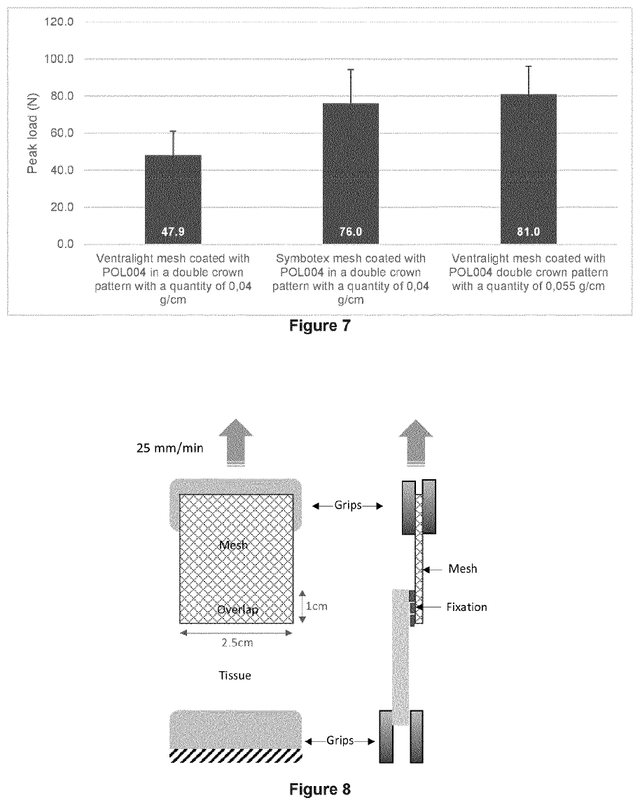

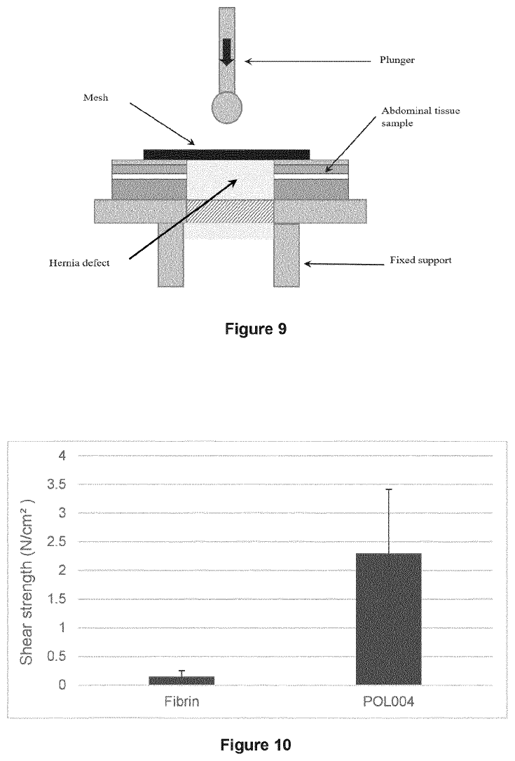

FIG. 7 illustrates burst acute performance of surgical materials of the present disclosure in two polymer quantity ratios. The set up illustrated in FIG. 9 was used for the test.

### Example 4. Usability Performance Test

### Symbotex™

Tests were conducted with the polymer composition, POL004, side by side, with the current standard of care Absorbatacks™ to fix a Symbotex™ mesh with POL004. Those tests were done on pig cadavers freshly sacrificed to be able to assess both polymer acute performance and usability. The pig model was considered as relevant to assess product usability because of its resemblance with the human.

A midline hernia was created and repaired (classically treated using laparoscopic IPOM approach) using either (1) POL004+Symbotex™ or (2) Absorbatacks™+Symbotex™.

Statements were then given by the experimentors (surgeons) to rate the solution from “I totally agree” giving 5 point to “I totally disagree” giving 1 point.

As expected, the main and very appreciated advantage of the polymer was found on the ease of use, as using the polymer improved (1) the ability to center the mesh over the defect due to POL004's “post-it” effect and coating pattern that provided visual landmarks (2) ability to flatten the mesh over the defect not creating penetrating fixation points that disrupt the continuity between the mesh and the tissue (3) the ability of mesh repositioning before light activation.

### Proceed®

A similar test was done for the Proceed® mesh using Securestraps® as a control. Their usability was improved using a polymer of the present invention when compared to tacks.

### Ventralight™ ST

A similar test was done for the Ventralight™ mesh using SorbaFix™ or SorbaFix™ with Echo 2 Positioning System® as a control.

Surgeons' hands-on test results for both meshes are presented in the table below. The table shows that the surgical material with the polymer composition, such as POL004, is easier to center, easier to obtain good mesh conformation to the wall, and easier to reposition, thus yielding an overall improvement of the overall surgical method. Grades including * mark in the table are based on interpretation from surgeons' feedback.

| | | | | | Ventralight ™ | | |
|---|---|---|---|---|---|---|---|
| | Symbotex ™ | | Proceed ® | | | | SorbaFix ™ with Echo 2 |
| Statements | POL004 | AbsorbaTacks ™ | POL004 | Securestraps ™ | POL004 | SorbaFix ™ | Positioning System |
| Rating scale: | Surgeon 1 | Surgeon 1 | Surgeon 1 | Surgeon 1 | Surgeon 4 | Surgeon 4 | Surgeon 4 |
| from 1 to 5, 5 | Surgeon 2 | Surgeon 2 | Surgeon 2 | Surgeon 2 | | | |
| being “I total | Surgeon 3 | Surgeon 3 | | | | | |
| agree with the | | | | | | | |
| statement” | | | | | | | |
| The mesh was | 5 | 5 | 3 | 3 | 3.5 | 5 | 1 |
| easily | 4.5 | 5 | 3 | 5 | | | |
| prepared | 5 | 5 | | | | | |
| Folding, | | | | | | | |
| insertion | | | | | | | |
| The mesh was | 4 | 4 | 2 | 2 | 4 | 5 | 5 |
| easily | 4* | 4* | 4 | NA | | | |
| delivered | 4* | 4* | | | | | |
| Unfolding, | | | | | | | |
| identification | | | | | | | |
| parietal side | | | | | | | |
| The mesh | 4 | 2.5 | 2 | 1 | 5 | 2.5 | 3 |
| fixation step | 4.8 | 2.2 | 5 | 1.5 | | | |
| was easy | 5 | 5 | | | | | |
| Centering, | | | | | | | |
| flatness, | | | | | | | |
| repositioning | | | | | | | |

### Example 5. Light Activating Test

Experimental work was conducted to conform that the polymer composition could be efficiently light-activated through the mesh in an optimal time, meaning the polymer was enough cross-linked to achieve its adhesive function, without extending the operative time.

The polymer composition was applied in a double crown pattern with dots in between and activated by a light source prototype for use in minimally invasive surgery. The light is composed of a 405 nm LED.

The polymer composition light activation through the mesh was assessed in two series of experiments.

First, the meshes' transmittance of 405 nm wavelength radiation was measured. The light intensities that are transmitted through the meshes when. The light source is at 1 cm distance are displayed in the table below:

| | Distance | |
|---|---|---|
| | (Light source/Mesh) | |
| | 1 cm | |
| Measurement location | Center | Edge |
| Control (no sample) | 564 | 170 |
| PP layer | 545 | 165.3 |
| Symbotex TM | 428 | 126 |
| | (τ = 7 sec) | (τ = 7 sec) |
| Proceed + PP layer | 209 | 62.5 |
| | (τ = 7 sec) | (τ = 13 sec) |
| Ventralight TM ST + PP layer | 322 | 91.7 |
| | (τ = 7 sec) | (τ = 9 sec) |

Second, the polymer composition cross-linking degree after light exposure was quantified using Fourier transform infrared (FTIR) spectroscopy to quantify the acrylate conversion.

As shown in FIG. 20, it was demonstrated that a high rate of acrylate conversion (>90%) can be achieved for the polymer composition when using different meshes. The time or intensity of polymerization will depend on the light transmittance properties of the mesh used, as well as the characteristics of the light source. As shown in FIG. 21(a)-(c), ratios and/or weight calculations may vary depending on the chosen mesh and/or polymer pattern.

### Preclinical Results

Different types of test can be run to assess the surgical material's product performance and compare it to the use of tacks (standard of care):

| Type of test | Tested parameter |
|---|---|
| Lap shear | Ex vivo fixation strength—representative of the shearing |
| (ex vivo) | movement of the viscera on the abdominal wall. |
| | FIG. 8 illustrates the set-up for lap shear testing. |
| Burst ball | Ex vivo fixation strength—representative of the bulging |
| (ex vivo) | of the viscera through the abdominal wall. FIG. 9 |
| | illustrates the set-up for burst ball testing. |
| Acute pig | Usability (Post-it effect, repositioning, easiness of mesh |
| cadaver | centering over defect, time needed for mesh placement), |
| | qualitative fixation strength |
| Acute human | Usability in different anatomies. |
| cadaver | |
| Chronic pig | All these endpoints were evaluated after 3 months mesh |
| study | implantation: Mesh centering over defect, mesh |
| | migration, mesh shrinkage, chronic fixation strength, |
| | local tissue response, impact on mesh cell ingrowth, |
| | adhesion formation |

### Pre-Clinical Test 1. Acute Ex Vivo Lap Shear Performance

As illustrated in FIG. 10, acute ex-vivo tests have been performed to compare POL004's performance to a fibrin product to fix Symbotex™ meshes. Lap shear method was used. The test results are shown in FIG. 10.

### Pre-Clinical Test 2. Acute Ex Vivo Burst Ball Performance

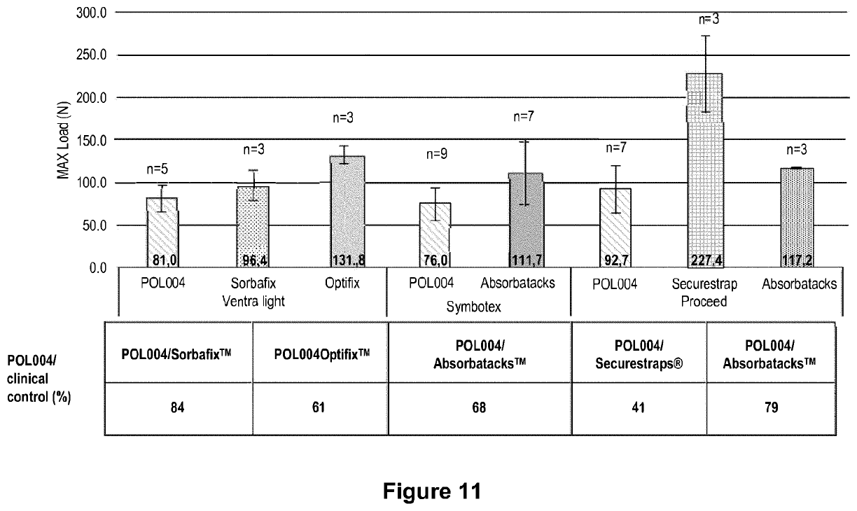

As illustrated in FIG. 11, the graph below displays a polymer composition's burst ball performance for the three commercial meshes in comparison to the fixation standard of care (tacks).

Securestraps® display the highest performance of all commercial products. Overall, hernia mesh products of the present disclosures resulted in 61 to 84% of tacks performance, except for Securestraps®. These results should be compared with the maximal abdominal pressure sustained by the human abdominal wall. This pressure reaches 170 mmHg (2N/cm²) during coughing or jumping. For a defect of ˜12 cm² (4 cm defect diameter) the mesh should at least sustain a load equal to ˜25N. This suggest that POL004 presents sufficient fixation strength to maintain a mesh over a defect until the wall is repaired.

### Pre-Clinical Test 3. Chronic Performance for the Symbotex™ Mesh

Chronic studies were performed using a porcine model with midline hernia, which is 3-4 cm excisional defect, intact peritoneum.

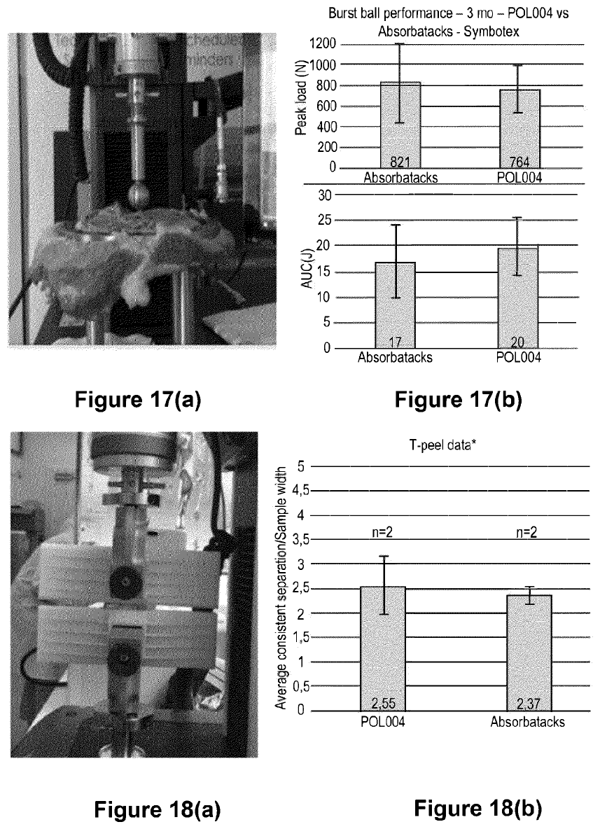

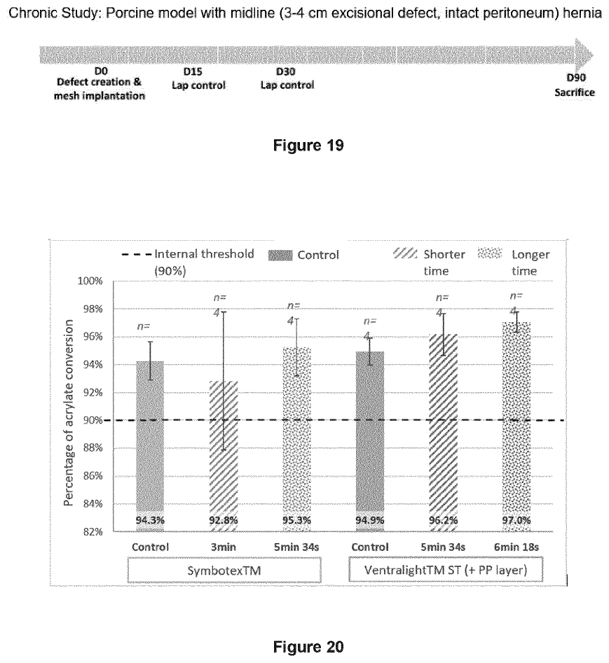

As shown in FIG. 17(b), based on the burst ball performance results of the 3-month chronic study, the surgical material including POL004 showed equivalent performance to Absorbatacks™ to fix a Symbotex™ mesh with respect to the shown properties. Similar result has been obtained with Ventralight™ mesh as shown in FIG. 22, illustrating POL004 showed equivalent or superior performance to SorbaFix™.

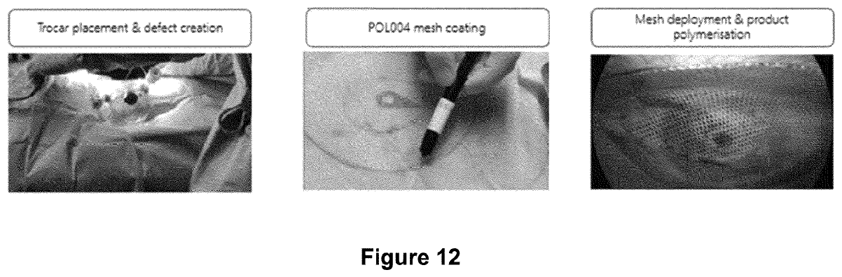

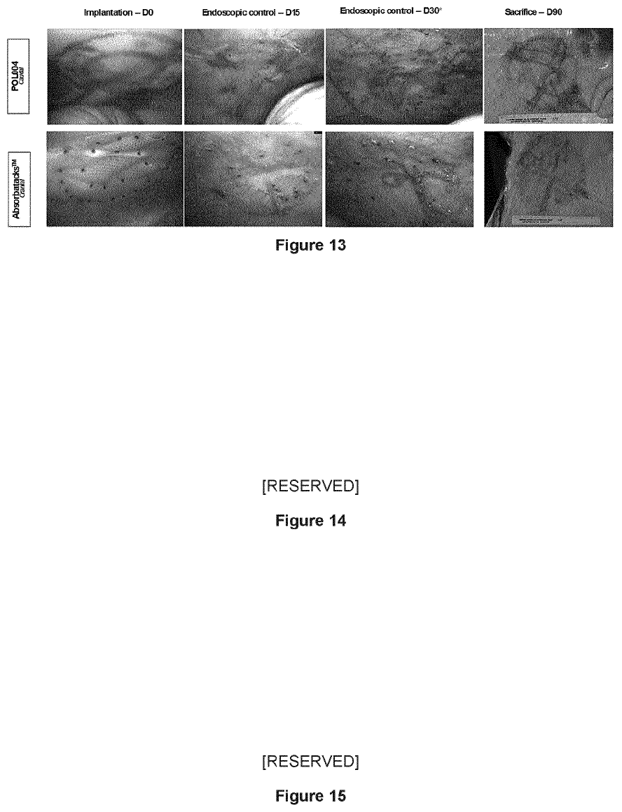

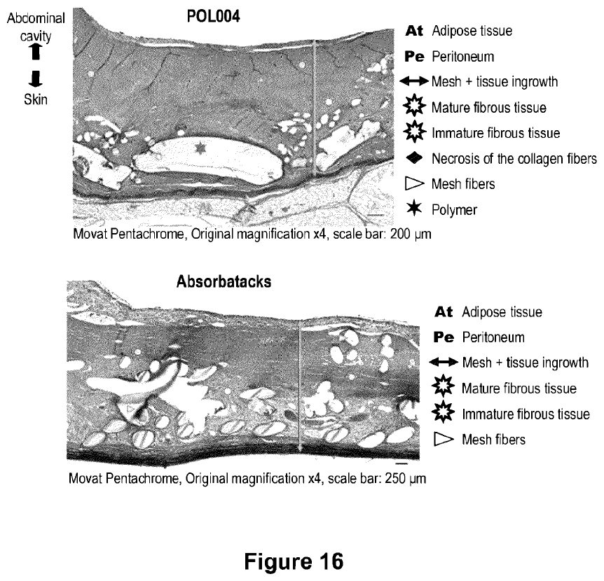

Details of the chronic studies are illustrated in FIGS. 12 through 18. FIG. 12 illustrates the chronic study's design in three steps. FIG. 13 illustrates appearance of tested meshes centered at different timepoints up to three months. FIG. 16 illustrates the tissue tolerance and ingrowth of the surgical material, compared to the control after 3 months implantation. Method includes: n=2 animals per group, using midline model, Movat Pentachrome staining. FIG. 16 illustrates that: (1) Mild tissue ingrowth can be observed in all implanted site independently of the fixation technique (2) For both groups, fibrous tissue was diffuse in the whole mesh (3) Local tolerance of POL004 at both timepoints was considered as excellent.

FIG. 17(a) illustrates a burst ball set up. FIG. 17(b) illustrates burst ball test results at three months of a surgical disclosure of the present invention compared to Absorbactacks™. Mechanical tester used is (1) Instron (2) 5 kN load cell (3) compression at a rate of 25.4 mm/min. Burst ball setting is, (1) upper jaw is 15 cm diameter in the middle, (2) plunger is 2.54 cm diameter, and (3) 8 penetrating screws to fix the tissue on the setting. For both conditions shown in FIG. 17(b), mesh and tissue were pierced before mesh-tissue interface failure.

FIG. 18(a) illustrates a T-peel test set up. FIG. 18(b) illustrates ingrowth strength of a surgical material of the present invention compared to Absorbactacks™. Mechanical tester used is (1) Instron (2) 500N load cell (3) compression at a rate of 25 mm/min. Tissue used is (1) fresh pig abdominal wall, (2) 1 muscle layer remining, and (3) 6×2 cm after three months of ingrowth. As illustrated in FIG. 18(b), results indicate similar ingrowth strength between the POL004 and the Absorbactacks™ group.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

### I. Bibliography

[1] Funk, L. M. (2016). Laparoscopic ventral hernia repair. In Illustrative Handbook of General Surgery: Second Edition (pp. 555-566). https://doi.org/10.1007/978-3-319-24557-7_34
[2] Gungor, B., Malazgirt, Z., Topgül, K., Gök, A., Bilgin, M., & Yürüker, S. (2010). Comparative Evaluation of Adhesions to Intraperitoneally Placed Fixation Materials: A Laparoscopic Study in Rats. Indian Journal of Surgery, 72(6), 475-480. https://doi.org/10.1007/s12262-010-0168-3
[3] Karahasanoglu, T., Onur, E., Baca, B., Hamzaoglu, I., Pekmezci, S., Boler, D. E., . . . Altug, T. (2004). Spiral tacks may contribute to intra-abdominal adhesion formation. Surgery Today, 34(10), 860-864. https://doi.org/10.1007/s00595-004-2831-4
[4] Zihni, A. M., Cavallo, J. A., Thompson, D. M., Chowdhury, N. H., Frisella, M. M., Matthews, B. D., & Deeken, C. R. (2015). Evaluation of absorbable mesh fixation devices at various deployment angles. Surgical Endoscopy, 29(6), 1605-1613. https://doi.org/10.1007/s00464-014-3850-x
[5] Fortelny, R. H., Petter-Puchner, A. H., Walder, N. et al. Cyanoacrylate tissue sealant impairs tissue integration of macroporous mesh in experimental hernia repair. Surg Endosc 21, 1781-1785 (2007). https://doi.org/10.1007/s00464-007-9243-7
[6] Parker, Samuel G et al. “Nomenclature in Abdominal Wall Hernias: Is It Time for Consensus?” World journal of surgery vol. 41,10 (2017): 2488-2491. doi:10.1007/s00268-017-4037-0

What is claimed is:

1. A surgical material comprising a polymer composition applied to a mesh substrate, wherein the polymer composition has a post-it effect allowing the surgical material to be repositioned on body tissue during surgery and wherein the polymer composition can be activated after positioning on the body tissue to attach the surgical material to the tissue.

2. The surgical material according to claim 1, wherein the polymer composition comprises a poly(glycerol sebacate acrylate) or derivative thereof.

3. The surgical material according to claim 1, wherein the polymer composition is activated by light.

4. The surgical material according to claim 1, wherein the mesh is a circular shape, and wherein a ratio of a weight of the polymer composition to a diameter of the mesh is from about 0.04 g/cm to about 0.06 g/cm.

5. A method of treating a hernia comprising,
i) positioning a surgical material as claimed in claim 1 over a herniated defect, and wherein the surgical material preferably comprises a polymer composition comprising a poly(glycerol sebacate acrylate) or derivative thereof; and
ii) activating the polymer composition to attach the surgical material to tissue within a body proximate the herniated defect.

6. The method of treating a hernia according to claim 5, further comprising repositioning the surgical material as necessary, after step i) and before step ii).

7. The method of treating a hernia according to claim 5, wherein the polymer composition is activated by light during step ii).

8. A method of manufacturing a surgical material, comprising applying a polymer composition comprising a poly(glycerol sebacate acrylate) or derivative thereof on a mesh substrate, wherein the polymer composition can be activated during surgery to attache the surgical material to tissue within a body.
